(19) United States
(12) Plant Patent Application Publication
(10) Pub. No.: US 2006/0143757 P1
(43) Pub. Date: Jun. 29, 2006

(54) CHRYSANTHEMUM PLANT NAMED 'LIPSI'

(50) CHRYSANTHEMUM MORIFOLIUM; CHRYSANTHEMUM

(75) Inventor: Mark Roland Boeder, The Hague (NL)

Correspondence Address: PARKHURST & WENDEL, L.L.P., 1421 PRINCE STREET, SUITE 210, ALEXANDRIA, VA, 22314-2805, US

(73) Assignee: Chrysanthemum Breeders Association N.V., Aalsmeer (NL)

(21) Appl. No.: 11/020,471

(22) Filed: Dec. 27, 2004

(51) Int. Cl. A01H 5/00

(52) U.S. Cl. PLT/290

(57) ABSTRACT

A chrysanthemum plant named ‘Lipsi’ characterized by its medium sized blooms with salmon ray florets and prolific branching; natural season flower date August 23-30; blooming for a period of 5 weeks.

### BACKGROUND OF THE INVENTION

‘Lipsi’ is a product of a breeding and selection program for outdoor pot mums (garden mums) which had the objective of creating new chrysanthemum cultivars with a decorative type flower, a natural season flower date around August 23-30; blooming for a period of 5 weeks. The new plant of the present invention comprises a new and distinct cultivar of chrysanthemum plant ‘Lipsi’ is a seedling resulting from a crossing program, set up by a previous breeder, and which records are unknown to the inventor. The new and distinct cultivar was discovered and selected as a flowering plant by Mark Roland Boeder on a cultivated field in Rijsenhout, The Netherlands in 2001. The first act of asexual production of ‘Lipsi’ was accomplished when vegetative cuttings were taken from the initial selection in 2001 in a controlled environment in Rijsenhout, The Netherlands, and propagated further at this location. The new cultivar has been found to retain its distinctive characteristics through successive propagations.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

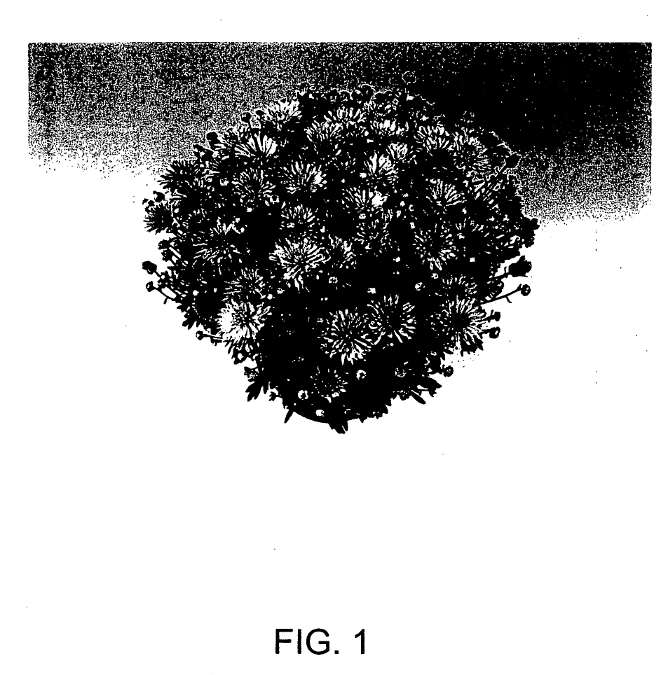

FIG. 2 shows the various stages of bloom of the new cultivar.

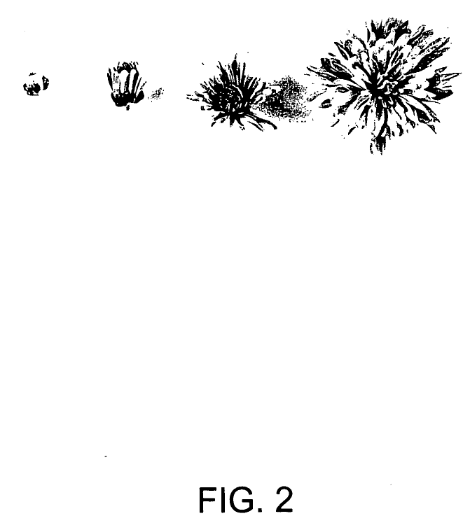

FIG. 3 shows the foliage of the new cultivar.

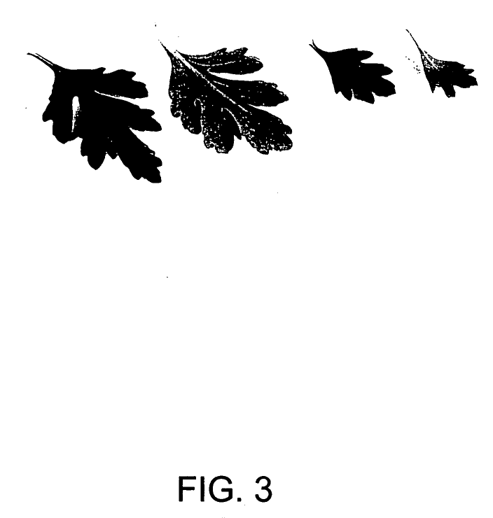

### DESCRIPTION OF THE INVENTION

This new variety of chrysanthemum is of the botanical classification Chrysanthemum morifolium L. The observations and measurements were gathered from plants grown out door in Rijsenhout, The Netherlands under natural day length and temperature and planted week 24 in 2004. The natural blooming date of this crop was August 23-30 (week 35). The average height of the plants was 25 cm. No growth retardants were used. No tests were done on disease or insect resistance or susceptibility. No tests were done on cold or drought tolerance. This new variety produces medium sized blooms with salmon ray florets blooming for a period of 5 weeks.

From the cultivars known to inventor the most similar existing cultivar in comparison to ‘Lipsi’ is ‘Stella’ (U.S. Plant Patent pending). When ‘Stella’ and ‘Lipsi’ are being compared the following differences are noticed: The differences of ‘Stella’ and ‘Lipsi’ are (1) Color ray-florets. The flowers of ‘Stella’ are more salmon colored than those of ‘Lipsi’, which have an orange hue in its soft colored florets. (2) Width ray-florets. The ray-florets of ‘Stella’ are wider than those of ‘Lipsi’. (3) Natural blooming date. Under natural conditions, ‘Lipsi’ flowers earlier than ‘Stella’.

The following is a description of the plant and characteristics that distinguish ‘Lipsi’ as a new and distinct variety. The color designations are taken from the plant itself Accordingly, any discrepancies between the color designations and the colors depicted in the photographs are due to photographic tolerances. The color chart used in this description is: The Royal Horticultural Society Colour Chart, edition 1995.

TABLE 1
Botanical Description of chrysanthemum plant ‘LIPSI’

| | |
|---|---|
| Bud | |
| Size | Small; cross-section 0.5 cm, height 0.7 cm |
| Outside Color | Yellow-green 145C |
| Involucral bracts | 2 rows, length 7 mm, width 3 mm |
| Involucral bracts | Not present |
| among disc-florets | |
| Involucral bracts | Green 138A |
| color | |
| Bloom | |
| Type | Decorative |
| Height | 1.5 cm |
| Size | Medium |
| Fully Expanded | 5.5 cm |
| Peduncle length | 9-12 cm |
| Peduncle color | Yellow-green 146C |
| Number of | Approx. 5-6 blooms per branch |
| blooms per branch | |
| Performance on | 5 weeks |
| the plant | |
| Seeds | Produced in small quantities, ovate grey-brown |
| | 199A, 1½ mm in length. |
| Fragrance | Typical chrysanthemum, slightly |
| Color | |
| Center of the flower | Immature Greyed-yellow 162C |
| | Mature Yellow-orange 23C |
| Color of upper | Greyed-orange 174D to Orange 24D |
| surface of the ray- | |
| florets | |
| Color of the lower | Greyed-orange 163D at base blending to Greyed- |
| surface of the | orange 173D at tip |
| ray-florets | |
| Tonality from | A garden mum with salmon flowers |
| Distance | |
| Discoloration | Greyed-orange 164D to Greyed-orange 173D |
| to color | |
| Ray florets | |
| Texture | Upper and under side smooth |
| Number | 160-180 |
| Cross-section | Flat |
| Longitudinal axis | Straight |
| of majority | |
| Length of | 0.3 cm |
| corolla tube | |
| Ray-floret margin | Entire |
| Ray-floret length | 2.5 cm |
| Ray-floret width | 0.4 cm |
| Ratio length/ | High |
| width | |
| Shape of tip | Pointed |
| Disc florets | |
| Disc diameter | 0.3-0.4 cm |
| Distribution of | Few, only visible in mature stage |
| disc florets | |
| Shape | Tubular |
| Color | Yellow 11D at base to Yellow-orange 23C at top |
| Receptacle shape | Conical raised |
| Reproductive | |
| Organs | |
| Stamen | Present in disc florets only |
| Stamen color | Yellow-green 144A |
| Pollen | Present |
| Pollen color | Yellow 7A |
| Styles | Thick |
| Style color | Yellow 13A |
| Style Length | 0.4 cm |
| Stigma color | Yellow-green 144A |
| Stigma Width | 1 mm |
| Ovaries | Enclosed in calyx |
| Plant | |
| Form | Grown as a spray type potmum, outdoor |
| | mounded and round |
| Growth habit | Spherical shape |
| Growth rate | Medium |
| Height | 25 cm |
| Width | 35 cm |
| Stem Color | Greyed-brown 199C |
| Stem Strength | Strong |
| Stem Brittleness | Not brittle |
| Stem Anthocyanin | Absent |
| Coloration | |
| Internode length | 3-4 cm |
| Length of | From top to bottom 18 cm |
| lateral branch | |
| Lateral branch | Yellow-green 146D |
| color | |
| Lateral branch, | Medium strength |
| attachment | |
| Branching (average | Good with 6-8 breaks after pinching |
| number of | |
| lateral branches) | |
| Natural season | August 23-30 |
| blooming date | |
| Foliage | |
| Leaf color | Upper side Green 139A |
| | Lower side Green 138A |
| Color midvein | Upper side Yellow-green 148D |
| | Lower side Yellow-green 147D |
| Size | Small.; length 4.5-6 cm, width 2-3.5 cm |
| Quantity (number | 15 |
| per lateral | |
| branch) | |
| Shape | Elliptic |
| Texture upper side | Glabrous |
| Texture under side | Pubescent |
| Venation arrangement | Palmate |
| Shape of the margin | Serrated |
| Shape of Base of | Rounded |
| Sinus Between | |
| Lateral Lobes | |
| Margin of Sinus | Diverging |
| Between Lateral | |
| Lobes | |
| Shape of Base | Acute |
| Apex | Mucronulate |
| Petiole length | 1-2 cm |
| Petiole color | Yellow-green 148D |

TABLE 2
Differences with the comparison varieties

| | ‘Lipsi’ | ‘Stella’ |
|---|---|---|
| Color upper surface | Greyed-orange 174D | Orange 29C |
| ray-florets | to Orange 24D | |
| Width ray-florets | 0.4 cm | 0.7 cm |
| Start blooming date | Week 35 | Week 38-39 |
| (planted in week 24) | | |

I claim:

1. A new and distinct variety of chrysanthemum plant as described and illustrated.
